(19) United States
(12) Patent Application Publication
(10) Pub. No.: US 2014/0144433 A1
(43) Pub. Date: May 29, 2014

(54) DOSIMETRIC THERAPEUTIC GAS DELIVERY SYSTEM FOR NITRIC OXIDE UTILIZATION MONITORING AND CONTROL

(71) Applicants: AMERICAN AIR LIQUIDE, INC., Fremont, CA (US); AIR LIQUIDE SANTE (INTERNATIONAL), Paris (FR)

(72) Inventors: Andrew Martin, Wilmington, DE (US); Ira Katz, Meudon (FR)

(73) Assignees: Air Liquide Sante (International), Paris (FR); American Air Liquide, Inc., Fremont, CA (US)

(21) Appl. No.: 13/687,253

(22) Filed: Nov. 28, 2012

(51) Int. Cl. A61M 16/10; A61M 16/00; A61M 16/12

(52) U.S. Cl. CPC A61M 16/104; A61M 16/12; A61M 16/0057; A61M 16/0051
USPC 128/202.22; 128/203.14

(57) ABSTRACT

The disclosure describes a technique for monitoring patient utilization of inhaled Nitric Oxide as well as waste exhaust of Nitric Oxide in gases exhaled from patient lungs. By monitoring the real dose provided to a patient, actual compliance with therapeutic target doses may be monitored to improve patient safety and therapeutic benefit from inhaled Nitric Oxide. Simultaneously, unnecessary waste of inhaled Nitric Oxide may be avoided thereby increasing the cost effectiveness of Nitric Oxide therapy. The minimization of Nitric Oxide waste has the further benefit of reducing environmental Nitrogen Dioxide levels in e.g. a NICU environment thereby mitigating medical personnel's Nitrogen Dioxide exposure.

### TECHNICAL FIELD

The field relates to the control of medical gas dosimetry and monitoring of excess medical gas waste.

### BACKGROUND ART

Current standard Nitric Oxide (“NO”) delivery devices control the concentration of NO delivered into a conduit carrying gas to the patient for inhalation (e.g. the inspiratory limb of a ventilator breathing circuit or other breathing-gas administration system). Monitoring of delivered time-averaged NO concentrations is also performed on inspired gases. Accordingly, such systems do not differentiate between a) NO that is efficiently transported to gas-exchange regions of the lung and absorbed into the capillary blood and b) NO which is ultimately exhaled and wasted. As a result, NO uptake may be significantly different from patient to patient, even when inhaled NO concentrations are equivalent. This complicates optimization of dosing and weaning, as well as strategies to avoid adverse effects, all of which are areas of ongoing work (see, e.g., Gentile, Respiratory Care. 2011; 56: 1341-1359). Further, comparisons between different devices for administering NO is made difficult, and innovations that would potentially reduce the consumption of NO required for treatment, as well as ambient exposure of healthcare workers to NO and nitrogen dioxide, have not been commercialized.

Numerous publications and patents exist pertaining to delivery of inhaled NO. These are summarized, for example, in U.S. Pat. No. 6,581,599 issued to Stenzler, and can be broadly sorted into the following categories:

Continuous Delivery

NO contained in a gas cylinder, typically at concentrations between 100 and 1000 ppmv in nitrogen, is delivered through a pressure regulator and control valve at a constant flow rate into the inspiratory limb of a breathing circuit. Such systems are simple, and if the flow of air and/or oxygen in the breathing circuit is also constant, they deliver a fixed concentration of NO to the patient (in the range of 1-100 ppm, and more typically 1-40 ppm). However, it is well-known (see, e.g., Imanaka et al, Anesthesiology. 1997; 86: 676-688) that when used with the majority of ventilators, for which flow in the inspiratory limb is zero or at least reduced during exhalation, continuous NO delivery results in large variation, in the form of sharp spikes or boluses, in inhaled NO concentrations. This unintentional variation is generally considered unfavorably, and certainly leads to measurement inaccuracies when inhaled NO concentrations are monitored with conventional, slow time-response electrochemical sensors.

Intermittent/Sequential Delivery

This technique evolved from continuous delivery to address the inaccuracies described above. Delivery of NO into the breathing circuit is sequenced to correspond with patient inspiration, and switched off during exhalation. However, when on, the delivery of NO is done at a constant flow rate. As a result, when the inspiratory flow rate is constant (i.e. a square wave pattern, as typically occurs for volume control ventilation), the inhaled NO concentration is constant, but when the inspiratory flow rate varies (as occurs for pressure control ventilation, or during spontaneous breaths), the inhaled NO concentration varies (see, e.g., Imanaka et al, Anesthesiology. 1997; 86: 676-688, or Mourgeon et al, Intensive Care Med. 1997; 23: 849-858). As for continuous delivery, intra-breath variation in inhaled NO concentration goes unnoticed when monitored with conventional, slow time-response sensors, and in such circumstances causes inaccuracies in the monitored concentration.

Proportional Delivery

Devices that deliver NO at flow rates that vary in proportion to the flow in the inspiratory limb of the breathing circuit are the current standard for NO administration systems. Inspiratory flow patterns are obtained directly from the ventilator, or through flow sensors inserted into the inspiratory limb, and the delivered flow rate of NO is adjusted proportionally so as to maintain a constant, or near-constant, inhaled NO concentration. Such systems have been described in numerous past publications, for example in Hiesmayr et al, Brit. J. Anaesthesia; 1998; 81: 544-552, and in Kirmse et al, Chest; 1998; 113: 1650-1657, and in several patents, for example in U.S. Pat. No. 5,558,083 issued to Bathe et al.

Pulsed/Bolus/Spiked Delivery

This category is made up of a family of techniques in which the inhaled NO concentration is deliberately varied over a single inhalation, and is most pertinent to the present invention. Generally, the intention is to target delivery of NO to preferred lung regions (e.g. the alveolar spaces) and limit delivery to non-preferred regions (e.g. the conducting airways). Examples may be found in publications by Katayama et al, Circulation. 1998; 98: 2129-2432, by Heinonen et al, Intensive Care Med. 2000; 26: 1116-1123, and in U.S. Pat. Nos. 5,839,433 6,581,599 and 6,694,969 issued to Higenbottam, Stenzler, and Heinonen, respectively. These techniques offer significant potential for improved dosing of NO; however, the traditional dose-metric of inhaled NO concentration is ill-suited to such approaches.

In an animal model, Heinonen et al evaluated a pulsed delivery technique by measuring changes in pulmonary arterial pressure with increasing NO dose, defined in terms of nanomoles NO delivered per minute. However, in this case the NO delivery represented the inhaled NO, and did not differentiate between NO absorbed into the capillary blood and NO that was exhaled. These authors do go on to write an equation for the NO uptake into the blood as:

NOᵤₚₜₐₖₑ=(∫F_{I}NO·\dot{V}_{I}·dt−∫F_{E}NO·\dot{V}_{E}·dt)·RR (1)

where F_{I}NO and F_{E}NO represent the inhaled and exhaled NO concentrations, respectively, V′_{I} and V′_{E} represent the inspiratory and expiratory flow rates, respectively, and RR represents the respiratory rate.

The method for determining NO uptake outlined in equation 1 suffers several drawbacks. First, it requires that NO concentrations and flow rates be known a priori or measured in both the inspiratory and expiratory flow. Second, it does not account for NO that reacts with O₂ and is subsequently exhaled as NO₂. In the accounting described by equation (1), such NO would be erroneously included as uptake. Third, as defined by Heinonen et al, V′_{E} is the flow rate, and F_{E}NO the NO concentration, of gas exhaled by the patient. This makes monitoring F_{E}NO difficult when using a ventilator with expiratory bypass flow, as in such cases the expiratory branch of the breathing circuit may contain both gas exhaled by the patient and gas passing directly from the inspiratory branch.

The problem addressed by the invention therefore is the administration of nitric oxide (NO) to a patient with the desired dosage of NO specified as the rate of uptake of NO into the capillary blood, expressed in units of mass, volume, or moles per unit time. Additionally, the solution preferably includes a way to monitor the uptake of NO in such a manner as to distinguish NO that is taken up into the blood from that which is exhaled and wasted. Accordingly, the medical practitioner administering NO may adjust dosing parameters so as to achieve a desired, known rate of uptake regardless of patient specific variation in, e.g., breathing pattern, minute volume, anatomical dead space and/or alveolar dead space.

The present invention therefore refines and improves NO dosing by controlling and monitoring the mass, volume, or molar uptake of NO, as well as monitoring NO wastage. This will allow users to better compare alternative NO delivery methods, and to titrate dosing to individual patients

### SUMMARY OF INVENTION

The invention may be understood in relation to the following embodiments listed as numbered sentences with internal cross referencing:
[1] An apparatus for delivering precise dosing of a Nitric Oxide containing gas for delivery by patient inhalation, the apparatus comprising:
   a) A ventilation apparatus configured to deliver an oxygen containing gas to a patient interface for inhalation,
   b) A Nitric Oxide delivery apparatus configured to inject a Nitric Oxide containing gas into the oxygen containing gas prior to the patient interface,
   c) An exhaust line configured to receive an exhaled gas from a patient and to transport the exhaled gas to an exhaust vent,
   d) An exhaust sampling line in fluid communication with the exhaust line prior to the exhaust vent and configured to receive a portion of a gas in the exhaust line comprising any exhaled gas,
   e) A first electrochemical cell in fluid communication with the exhaust sampling line and configured to receive at least part of the portion of the gas in the exhaust line comprising any exhaled gas and further configured to measure an amount of a Nitric Oxide in the sampling portion of the gas in the exhaust line comprising any exhaled gas.
[2] The apparatus for delivering precise dosing of a Nitric Oxide containing gas for delivery by patient inhalation of sentence 1, wherein the apparatus is configured to form a bypass flow of oxygen containing gas into the exhaust line during a patient exhalation.
[3] The apparatus for delivering precise dosing of a Nitric Oxide containing gas for delivery by patient inhalation of sentence 1 or 2, further comprising a NO₂ converter configured to receive an exhaust gas sample from the exhaust gas sampling line and configured to convert NO₂ molecules to Nitric Oxide molecules on a one-to-one basis.
[4] The apparatus for delivering precise dosing of a Nitric Oxide containing gas for delivery by patient inhalation of sentence 1, 2 or 3, further comprising a second electrochemical cell in fluid communication with the exhaust sampling line and configured to receive at least part of the portion of the gas in the exhaust line comprising any exhaled gas and further configured to measure an amount of a NO₂ in the sampling portion of the gas in the exhaust line comprising any exhaled gas.
[5] The apparatus for delivering precise dosing of a Nitric Oxide containing gas for delivery by patient inhalation of sentence 1, 2, 3 or 4, further comprising a computer specifically programmed or a microprocessor specifically configured to execute the following functions:
   a) Calculate a Nitric Oxide dose provided to a patient by the Nitric Oxide delivery apparatus,
   b) Calculate an amount of NOx in the gas in the exhaust line comprising any exhaled gas,
   c) Based on the values calculated in step a) and step b), calculate an amount of Nitric Oxide absorbed by a patient.
[6] The apparatus for delivering precise dosing of a Nitric Oxide containing gas for delivery by patient inhalation of sentence 5, wherein the computer specifically programmed or a microprocessor specifically configured to execute functions b) and c) based on the following calculations:

$${\overline{\dot{m}}}_{NO , waste} = \frac{{\int }_{t}^{{t}^{′}} ( {C}_{NO} + {C}_{{NO}_{2}} ) \cdot {ρ}_{NO} \cdot {Q}_{E} d t}{T}$$

$$and$$

$${Uptake}_{NO} = {\overline{\dot{m}}}_{NO , del} - {\overline{\dot{m}}}_{NO , waste} .$$

[7] The apparatus for delivering precise dosing of a Nitric Oxide containing gas for delivery by patient inhalation of sentence 1, 2, 3, 4, 5 or 6, further comprising a positive expiratory pressure system comprising an exhalation valve and an exhalation pressure sensor in the exhaust line.
[8] The apparatus for delivering precise dosing of a Nitric Oxide containing gas for delivery by patient inhalation of sentence 3, 4, 5, 6 or 7, wherein the a NO₂ converter configured to receive an exhaust gas sample from the exhaust gas sampling line and configured to convert NO₂ molecules to Nitric Oxide molecules on a one-to-one basis comprises one or more of a thermal converter, a catalytic converter, and a reducing converter.
[9] The apparatus for delivering precise dosing of a Nitric Oxide containing gas for delivery by patient inhalation of sentence 1, 2, 3, 4, 5, 6, 7 or 8, wherein the exhaust line configured to receive an exhaled gas from a patient and to transport the exhaled gas to an exhaust vent further comprises a positive expiratory pressure system comprising an exhalation valve and an exhalation pressure sensor both in the exhaust line and further comprising an exhaust line flow sensor configured to measure a flow rate of the gas in the exhaust line.
[10] An apparatus for delivering precise dosing of a Nitric Oxide containing gas for delivery by patient inhalation, the apparatus comprising:
   a) A ventilation apparatus configured to deliver an oxygen containing gas to a patient interface for inhalation, the ventilation apparatus comprising,
      A) A source of medical air,
      B) A source of medical oxygen
      C) An oxygen containing gas injection device in fluid communication with the source of medical air via a medical air supply line and the source of medical oxygen via a medical oxygen supply line,
      D) One or more medical air pressure regulators in fluid communication with the medical air supply line and configured to control the pressure of the medical air in the medical air supply line,
      E) One or more medical oxygen pressure regulators in fluid communication with the medical oxygen supply line and configured to control the pressure of the medical oxygen in the medical oxygen supply line,
      F) A medical oxygen working pressure sensor configured to measure the pressure of a medical oxygen dose emitted from the oxygen containing gas injection device,
      G) A medical air working pressure sensor configured to measure the pressure of a medical air dose emitted from the oxygen containing gas injection device,
      H) An medical oxygen flow sensor configured to measure a flow rate of the medical oxygen dose emitted from the oxygen containing gas injection device,
      I) An medical air flow sensor configured to measure a flow rate of the medical air dose emitted from the oxygen containing gas injection device,
      J) An inspiratory gas tube in fluid communication with the oxygen containing gas injection device and configured to receive an injection of oxygen containing gas from the oxygen containing gas injection device,
      K) A patient circuit pressure sensor configured to measure a gas pressure in the inspiratory gas tube,
   b) A Nitric Oxide delivery apparatus configured to inject a Nitric Oxide containing gas into the oxygen containing gas prior to the patient interface, wherein the Nitric Oxide delivery apparatus comprises
      A) A Nitric Oxide dose control system configured to inject a controlled amount of Nitric Oxide into the oxygen containing gas,
   c) An exhaust line configured to receive an exhaled gas from a patient, a bypass flow of oxygen containing gas, or both, and to transport the exhaled gas to an exhaust vent,
   d) A positive expiratory pressure system comprising an exhalation valve and an exhalation pressure sensor in the exhaust line,
   e) An exhaust sampling line in fluid communication with the exhaust line prior to the exhaust vent and configured to receive a portion of a gas in the exhaust line comprising any exhaled gas,
   f) A first electrochemical cell in fluid communication with the exhaust sampling line and configured to receive at least part of the exhaust gas sample and further configured to measure the amount of Nitric Oxide in the sampling portion of the exhaled gas,
   g) A second electrochemical cell in fluid communication with the exhaust sampling line and configured to receive at least part of the exhaust gas sample and further configured to measure the amount of NO₂ in the sampling portion of the exhaled gas,
   h) An exhaust line flow sensor configured to measure a flow rate of the gas in the exhaust line,
   i) A patient interface in fluid communication with the inspiratory gas tube and the exhaust line,
   j) A computer specifically programmed or a microprocessor specifically configured to execute the following functions:
      A) Calculate a Nitric Oxide dose provided to a patient by the Nitric Oxide delivery apparatus,
      B) Calculate an amount of NOx in the gas in the exhaust line comprising any exhaled gas,
      C) Based on the values Calculated in A) and B), calculate an amount of Nitric Oxide absorbed by a patient.
[11] The apparatus of sentence 10, wherein the computer is further specifically programmed or a microprocessor is further specifically configured to calculate an amount of NO delivered to a patient based on the Nitric Oxide dose and the amount of NOx in the gas in the exhaust line.
[12] The apparatus of sentence 10 or 11, further comprising an exhaust gas sample mixing chamber between and in fluid communication with a) the exhaust sample line and b) the first and second electrochemical cells, wherein the exhaust gas sample mixing chamber is configured to form a substantially homogenous exhaust gas sample.
[13] The apparatus of sentence 10, 11 or 12, further comprising an alarm system configured to be activated in response to a calculated amount of NOx in the exhaust line gas in excess of a pre-defined threshold amount.
[14] The apparatus of sentence 10, 11, 12 or 13, further comprising an exhaust gas mixing chamber between and in fluid communication with a) the exhaust line and b) the exhaust sampling line, wherein the exhaust gas mixing chamber is configured to form a substantially homogenous exhaust gas upstream from the exhaust sampling line.

### DISCLOSURE OF INVENTION

An example general concept configuration is displayed schematically in FIG. 1. A cylinder (1) or other gas source supplies NO-containing gas (typically with NO concentration between 100 and 1000 ppm in nitrogen) through a pressure regulator (2) to the NO supply line (3) of the apparatus (15). The NO supply line carries the NO-containing gas to the administration block (4), which is controlled by the administration CPU (5). The administration CPU receives the desired NO dose from a user interface (6), and receives information (13) sent from a ventilator or other breathing gas delivery device, and/or from a flow sensor positioned in a conduit supplying breathing gas to a patient, describing, for example, the flow rate of breathing gas delivered to the patient, the volume of gas delivered to the patient per breath, and/or the timing of cycling between inspiration and expiration. Based on this information and the desired NO dose, the administration CPU controls the timing and positions of a system of one or more valves and/or switches contained in the administration block so as to administer a flow of NO-containing gas through an administration line (7) to a patient breathing circuit or other conduit carrying breathing gas to the patient (9). The flow of NO-containing gas may be constant, intermittent, pulsed, or otherwise varied according to the NO dosing strategy. External to the administration block, a flow sensor (8) is positioned in the administration line to measure the variation in the rate of flow of NO-containing gas with time. This information is sent to a monitoring CPU (10), which also receives the concentration of NO in the NO-containing gas from the user interface. Optionally, the concentration of oxygen is also sent to the monitoring CPU (10). From this information the monitoring CPU (10) calculates the delivered flux of NO in terms of mass, volume, or moles NO per unit time. Using the concentration of oxygen, the monitoring CPU (10) may also be programmed to calculate an estimated amount of NO₂ production.

Concurrently, a continuous sample of exhaled gas (12) is drawn into the apparatus (15) to a gas analysis block (11). Gas is sampled from a position in the expiratory portion of the breathing circuit through which passes gas exhaled by the patient as well as any gas from the inspiratory portion of the circuit that bypasses the patient. The gas analysis block contains electrochemical cell based sensors to measure the concentrations of NO and NO₂ in the sampled gas. This information is sent to the monitoring CPU (10). Additionally, the monitoring CPU receives information (14) sent from a ventilator or other breathing gas delivery device, or from a flow sensor positioned at or near the location of gas sampling, which describes the flow rate of gas through the expiratory portion of the breathing circuit. From this information, the monitoring CPU calculates the waste flux of NO in terms of mass, volume, or moles NO per unit time.

Finally, the monitoring CPU (10) calculates the NO uptake in terms of mass, volume, or moles NO per unit time by subtracting the waste flux of NO from the delivered flux of NO. The delivered flux of NO, the waste flux of NO, and the NO uptake are sent from the monitoring CPU to the user interface, where they may be displayed.

### BRIEF DESCRIPTION OF DRAWINGS

FIG. 1 schematically outlines an example configuration of apparatus for dosimetric administration and monitoring of NO.

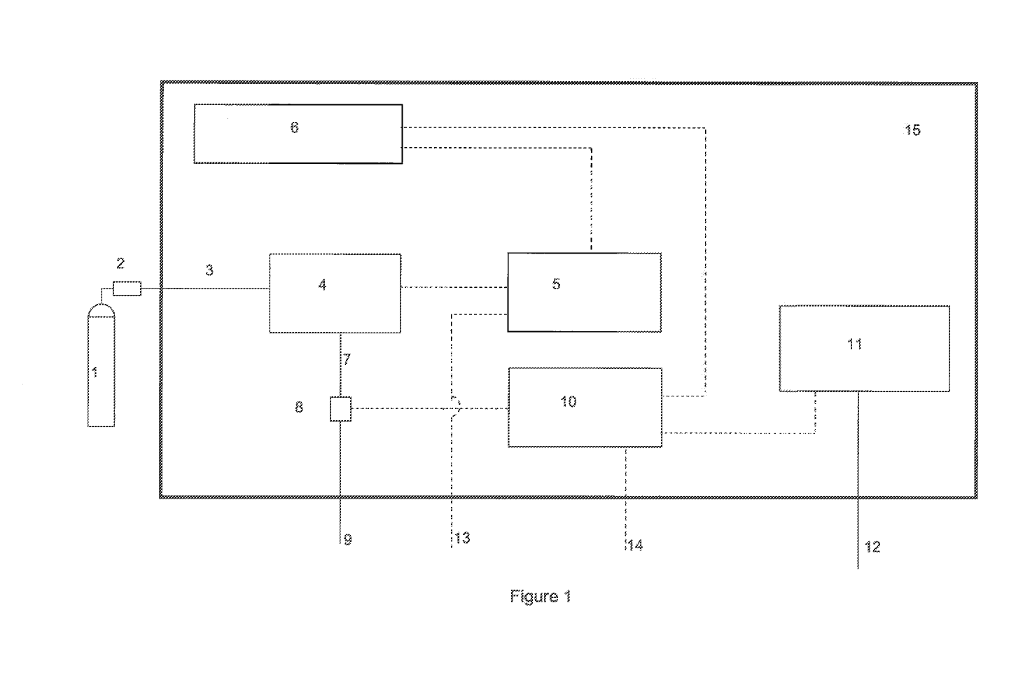

FIG. 2 is a more detailed schematic of a delivery system incorporating a preferred embodiment of the invention.

### MODE(S) FOR CARRYING OUT THE INVENTION

Concentrations of NO and NO₂ in gas sampled from the expiratory branch are determined using two or more electrochemical cells adapted to measure NO and NO₂. The waste flux of NO may be expressed in terms of mass, volume, or moles NO per unit time—if, for example, the waste flux is expressed in terms of mass, the following calculation is made:

$${\overline{\dot{m}}}_{NO , waste} = \frac{{\int }_{t}^{{t}^{′}} ( {C}_{NO} + {C}_{{NO}_{2}} ) \cdot {ρ}_{NO} \cdot {Q}_{E} d t}{T} \qquad ( 2 )$$

where C_{NO} and C_{NO2} are the concentrations of NO and NO₂ in the sampled gas as determined by their respective electrochemical cells; ρ_{NO} is the density of NO (at 1 atmosphere and at the temperature of gas in the expiratory branch of the breathing circuit, which may be assumed, e.g., as 36.6 degrees C., but is preferably measured, or acquired from the ventilator or breathing gas delivery device), and Q_{E} is the total volumetric gas flow rate through the expiratory branch of the breathing circuit. T is a time period starting at time t and ending at time t′.

Due to the slow time-response of the electrochemical cells, the waste flux and monitored NO uptake may not be calculated and displayed on a breath-by-breath basis. Rather, values determined for two or more breaths such as at least five breaths in sequence (preferably at least ten breaths in sequence) are averaged before being displayed.

The waste flux may be thus calculated and displayed based on averaged values determined for several breaths in sequence, and the average flux used in subsequent calculations and/or displayed.

The delivered flux of NO may be expressed in terms of mass, volume, or moles NO per unit time—if, for example, the delivered flux is expressed in terms of mass, the following calculation is made:

$${m}_{NO , del} = {\int }_{t}^{{t}^{′}} {C}_{NO} \cdot {ρ}_{NO} \cdot {Q}_{NO / {N}_{2}} d t \qquad ( 2 )$$

where, m_{NO, del} is the delivered mass of NO, C_{NO} is the concentration of NO in the supplied NO-containing gas (typically between 100 and 1000 ppmv in nitrogen, and preferably 800 ppmv), ρ_{NO} is the density of NO (at 1 atmosphere and at the temperature of the supplied NO-containing gas, which may be assumed, e.g., as 20 degrees C., or may be measured at flow sensor (8)) and Q_{NO/N2} is the administered volumetric flow rate of the NO-containing gas.

The delivered flux is then expressed as:

$${\overline{\dot{m}}}_{NO , del} = \frac{{m}_{NO , del}}{T} \qquad ( 3 )$$

where T is the same time period starting at time t and ending at time t′ as described above for the waste flux of NO.

The monitored NO uptake is then calculated as:

Uptake_{NO}={dot over ( m̅_{NO, del}−{dot over ( m̅_{NO, waste} (4)

The monitored NO uptake may be calculated and displayed based on averaged values determined for several breaths in sequence, and the average flux used in subsequent calculations and/or displayed.

Normally, the user interface will simultaneously display the target NO uptake (input by the user), the delivered flux, the waste flux, and the monitored NO uptake. Alarms may be activated when for example the waste flux becomes non-negligible or exceeds a threshold value based on the delivered flux and monitored NO uptake to alert the user that NO dosing is being performed inefficiently, that uptake has dropped below a therapeutic level, etc.

To ensure that potential transient variation in the NO and/or NO₂ concentration in the sampled gas does not cause inaccuracies in determining the waste flux of NO, a mixing element may be positioned in the expiratory line between the Y-piece and the sampling point, preferably immediately upstream from the sampling point. The mixing element may be as simple as a mixing chamber of sufficient internal volume that transient variations in NO and/or NO₂ in incoming gas are damped through the process of mixing with gas inside the chamber, such that these transient variations are absent or reduced to acceptably low amplitudes in gas exiting the chamber. Such a chamber would typically require an internal volume of on the order of the tidal volume of the patient e.g. between 0.5 and 1.0 liters for most adult patients. The electrochemical cell technology lacks the ability to deliver real time responsiveness. Nonetheless, sustained and consistent failures to reach therapeutic levels of uptake, over-dosing, etc., will be detected allowing for safer, more efficient and more effective NO administration based on patient specific data.

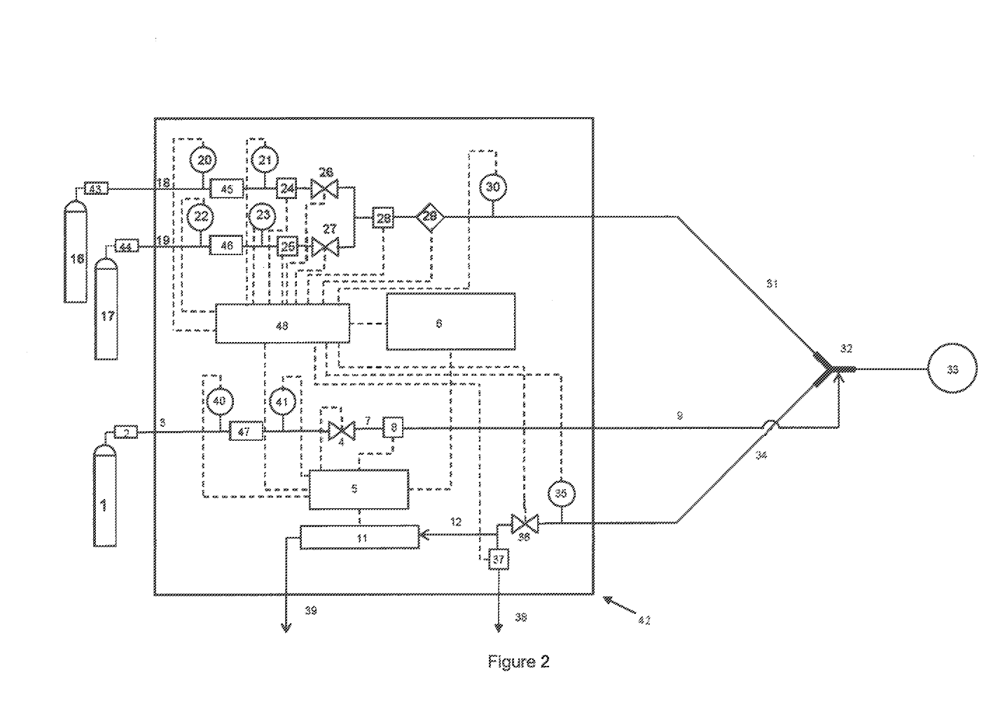

FIG. 2 shows a more detailed schematic of an example of the preferred embodiment. The numbered Figure elements are:

1—source of gas mixture containing therapeutic gas and carrier (e.g. 800-2000 ppm NO in balance N₂; but also could be CO, H₂ or H₂S in balance nitrogen, balance medical air, or balance inert noble gas such as helium or argon)

2—therapeutic gas supply regulator (delivery pressure, e.g., 3-6 bar)

3—therapeutic gas supply line

4—one or more control valves or switches used to supply therapeutic gas at a desired rate

5—therapeutic gas dosing CPU(s); may also include monitoring CPU(s) 10

6—GUI

7—therapeutic gas administration line

8—therapeutic gas line flow sensor

9—external therapeutic gas administration line connecting to breathing circuit distal to Y-piece

10—optional separate monitoring CPU (may be combined with administration CPU(s) 5)

11—therapeutic gas analysis block (electrochemical cells adapted to measure Nitric Oxide and/or Nitrogen Dioxide levels)

12—sample line from expiratory flow

16—source of medical air

17—source of medical oxygen

18—medical air supply line

19—medical oxygen supply line

20—pressure sensor for medical air supply pressure

21—pressure sensor for medical air working pressure

22—pressure sensor for medical oxygen supply pressure

23—pressure sensor for medical oxygen working pressure

24—medical air flow sensor

25—medical oxygen flow sensor

26—medical air control valve

27—medical oxygen control valve

28—mixed breathing gas flow sensor

29—oxygen sensor

30—low pressure sensor to measure pressure delivered to patient from ventilator (in cm H₂O)

31—inspiratory limb of breathing circuit

32—Y-piece

33—patient interface (endotracheal tube; facemask; hood; nasal mask/cushion/pillow/cannula)

34—expiratory limb of breathing circuit

35—low pressure sensor to measure positive expiratory pressures (in cm H₂O) (optional)

36—expiratory control valve

37—expiratory flow sensor

38—exhaust of breathing gases to atmosphere

39—exhaust of sample line gases to atmosphere

40—pressure sensor for therapeutic gas supply pressure

41—one or more pressure sensors for therapeutic gas working pressures on one or more dosing lines

42—Reference for the entire System

43—medical air supply regulator (with delivery pressure, e.g., 3-6 bar)

44—medical oxygen supply regulator (with delivery pressure, e.g., 3-6 bar)

45—medical air regulator (to more precisely control working pressure)

46—medical oxygen regulator (to more precisely control working pressure)

47—one or more therapeutic gas regulators to fix working pressure on one or more dosing lines

48—ventilation CPU

### DEFINITIONS AND/OR EXAMPLES:

Ventilation apparatus—ventilation apparatuses are established and widespread medical technology designed to support or substitute for a patient's physiological breathing. An overview of ventilation apparatuses encompassed within this definition is EDUARDO MIRELES-CABODEVILA, ENRIQUE DIAZ-GUZMAN, GUSTAVO A. HERESI, and ROBERT L. CHATBURN, Alternative modes of mechanical ventilation: A review for the hospitalist, Cleveland Clinic Journal of Medicine 2009; 76(7):417-430; doi:10.3949/ccjm.76a.08043.
Oxygen containing gas—An oxygen containing gas is any gas or gas mixture comprising or consisting of oxygen and medically suitable for administration to a patient. This includes medical oxygen meeting all applicable U.S. Food & Drug Administration requirements. See, e.g., CPG Sec. 435.100 Compressed Medical Gases—Warning Letters for Specific Violations Covering Liquid and Gaseous Oxygen, FDA, issued Nov. 5, 1987, revised Aug. 31, 1992; FDA, COMPRESSED MEDICAL GASES GUIDELINE (REVISED) February 1989. The oxygen concentration in a gas mixture may be for example anywhere from 21-100% and generally is adjusted based on blood oxygen saturation levels of a patient.
Positive expiratory pressure system—Positive expiratory pressure is also referred to as Positive end-expiratory pressure (PEEP). A positive expiratory pressure means a lung gas pressure above atmospheric pressure. A Positive expiratory pressure system is a device configured to ensure PEEP by artificially pressurizing the lungs. This is referred to as applied or extrinsic PEEP support. Generally a positive expiratory pressure system in the context of this invention is a subcomponent of a ventilation apparatus. Positive expiratory pressure systems within the scope of this term are described in “Mechanical Ventilation”, by Ryland P Byrd Jr, MD and Thomas M Roy, MD, accessed on <emedicine.medscape.com/article/304068-overview#aw2aab6b5>, last updated: Apr. 26, 2012.
Medical oxygen—This includes medical oxygen meeting all applicable U.S. Food & Drug Administration requirements. See, e.g., CPG Sec. 435.100 Compressed Medical Gases—Warning Letters for Specific Violations Covering Liquid and Gaseous Oxygen, FDA, issued Nov. 5, 1987, revised Aug. 31, 1992; FDA, COMPRESSED MEDICAL GASES GUIDELINE (REVISED) February 1989.
Medical air—means air that complies with one or more of the following standards:
   U.S. Food & Drug Administration requirements in CPG Sec. 435.100 Compressed Medical Gases—Warning Letters for Specific Violations Covering Liquid and Gaseous Oxygen, FDA, issued Nov. 5, 1987, revised Aug. 31, 1992; FDA, COMPRESSED MEDICAL GASES GUIDELINE (REVISED) February 1989;
   Medical air criteria defined in the current U.S. Pharmacopeia;
   The definition of Medical Air Quality from National Fire Protection Association 99, Standard for Health Care Facilities, 2005 edition, section 5.1.3.5.1.
Patient interface for inhalation—This is defined as any device adapted to deliver a medical gas for inhalation by a patient. There are many types of patient interfaces for inhalation including intubation tubes used in many mechanical ventilation situations, nasal cannula, and medical face masks. The choice of patient interface for inhalation depends on several factors such as the therapeutic purpose of the medical gas and the form of medical gas delivery. In the context of ventilation apparatus delivery of oxygen containing gases comprising Nitric Oxide, the most common choices are intubation tubes and nasal cannula.
Nitric Oxide delivery apparatus—These are medical devices designed to provide medically relevant doses of Nitric Oxide. Such devices may operate in a stand alone fashion or in conjunction with a ventilation apparatus. Nitric Oxide delivery apparatuses include but are not limited to those meeting the criteria defined by
   the U.S. Food and Drug Administration's Guidance Document for Premarket Notification Submissions for Nitric Oxide Delivery Apparatus, Nitric Oxide Analyzer and Nitrogen Dioxide Analyzer, issued Jan. 24, 2000; or
   European Committee for Standardization—CEN/TS 14507-1:2003 Inhalational nitric oxide systems—Part 1: Delivery systems 93/42/EEC (No).
Electrochemical cell—Electrochemical cells adapted to measure gaseous NO, NO2, or both, in a gas sample are disclosed for example in U.S. Pat. No. 4,052,268. Electrochemical cells in general should have the performance characteristics required for use in conjunction with Nitric Oxide delivery apparatuses as defined for example in U.S. Food and Drug Administration's Guidance Document for Premarket Notification Submissions for Nitric Oxide Delivery Apparatus, Nitric Oxide Analyzer and Nitrogen Dioxide Analyzer, sections 3.2 and 3.3, issued Jan. 24, 2000.
Nitric Oxide containing gas—means a gas comprising Nitric Oxide that is medically suitable for inhalation by a patient. Medical suitability includes but is not limited to a gas comprising Nitric Oxide that is a bioequivalent of the Nitric Oxide gas drug submitted under NDA 20845 and approved under U.S. Food and Drug Administration. Nitric Oxide containing gases include but are not limited to concentrated Nitric Oxide source gases and dilutions thereof. Concentrated Nitric Oxide containing gases are most commonly Nitric Oxide at a concentration of 100 ppm to 5000 ppm in a balance of U.S.P. Nitrogen gas. The FDA approved Nitric Oxide containing gases are 100 ppm and 800 ppm Nitric Oxide in a balance of U.S.P. Nitrogen gas.
NOx—means Nitric Oxide (NO) and Nitrogen Dioxide (NO₂).
NO₂ converter—Is a device adapted to quantitatively convert NO₂ to Nitric Oxide. NO₂ converters may be, for example, a thermal converter (>650 degrees C./stainless steel; 450 degrees C./Molybdenum), a catalytic converter (generally an Ag catalyst), or a reducing converter (reducing agents used include ascorbic acid).
Computer specifically programmed—means a general purpose programmable computer with specific software written to a component thereof such as a RAM component. Specific software is software designed to execute particular functions such as operating a robotic arm to carry out a manufacturing step or performing specific calculations or data transformations.
Microprocessor specifically configured—means an integrated circuit that is structurally designed to execute particular functions such as operating a robotic arm to carry out a manufacturing step or performing specific calculations or data transformations.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. An apparatus for delivering precise dosing of a Nitric Oxide containing gas for delivery by patient inhalation, the apparatus comprising:
a) a ventilation apparatus configured to deliver an oxygen containing gas to a patient interface for inhalation,
b) a Nitric Oxide delivery apparatus configured to inject a Nitric Oxide containing gas into the oxygen containing gas prior to the patient interface,
c) an exhaust line configured to receive an exhaled gas from a patient and to transport the exhaled gas to an exhaust vent,
d) an exhaust sampling line in fluid communication with the exhaust line prior to the exhaust vent and configured to receive a portion of a gas in the exhaust line comprising any exhaled gas,
e) a first electrochemical cell in fluid communication with the exhaust sampling line and configured to receive at least part of the portion of the gas in the exhaust line comprising any exhaled gas and further configured to measure an amount of a Nitric Oxide in the sampling portion of the gas in the exhaust line comprising any exhaled gas.

2. The apparatus for delivering precise dosing of a Nitric Oxide containing gas for delivery by patient inhalation of claim 1, wherein the apparatus is configured to form a bypass flow of oxygen containing gas into the exhaust line during a patient exhalation.

3. The apparatus for delivering precise dosing of a Nitric Oxide containing gas for delivery by patient inhalation of claim 1, further comprising a NO₂ converter configured to receive an exhaust gas sample from the exhaust gas sampling line and configured to convert NO₂ molecules to Nitric Oxide molecules on a one-to-one basis.

4. The apparatus for delivering precise dosing of a Nitric Oxide containing gas for delivery by patient inhalation of claim 1, further comprising a second electrochemical cell in fluid communication with the exhaust sampling line and configured to receive at least part of the portion of the gas in the exhaust line comprising any exhaled gas and further configured to measure an amount of a NO₂ in the sampling portion of the gas in the exhaust line comprising any exhaled gas.

5. The apparatus for delivering precise dosing of a Nitric Oxide containing gas for delivery by patient inhalation of claim 1, further comprising a computer specifically programmed or a microprocessor specifically configured to execute the following functions:
a) calculate a Nitric Oxide dose provided to a patient by the Nitric Oxide delivery apparatus,
b) calculate an amount of NOx in the gas in the exhaust line comprising any exhaled gas,
c) based on the values calculated in step f) and step g), calculate an amount of Nitric Oxide absorbed by a patient.

6. The apparatus for delivering precise dosing of a Nitric Oxide containing gas for delivery by patient inhalation of claim 5, wherein the computer specifically programmed or a microprocessor specifically configured to execute functions g) and h) based on the following calculations:
$${\overline{\dot{m}}}_{NO , waste} = \frac{{\int }_{t}^{{t}^{′}} ( {C}_{NO} + {C}_{{NO}_{2}} ) \cdot {ρ}_{NO} \cdot {Q}_{E} d t}{T}$$
$$and$$
$${Uptake}_{NO} = {\overline{\dot{m}}}_{NO , del} - {\overline{\dot{m}}}_{NO , waste} .$$

7. The apparatus for delivering precise dosing of a Nitric Oxide containing gas for delivery by patient inhalation of claim 1, further comprising a positive expiratory pressure system comprising an exhalation valve and an exhalation pressure sensor in the exhaust line.

8. The apparatus for delivering precise dosing of a Nitric Oxide containing gas for delivery by patient inhalation of claim 3, wherein the a NO₂ converter configured to receive an exhaust gas sample from the exhaust gas sampling line and configured to convert NO₂ molecules to Nitric Oxide molecules on a one-to-one basis comprises one or more of a thermal converter, a catalytic converter, and a reducing converter.

9. The apparatus for delivering precise dosing of a Nitric Oxide containing gas for delivery by patient inhalation of claim 1, wherein the exhaust line configured to receive an exhaled gas from a patient and to transport the exhaled gas to an exhaust vent further comprises a positive expiratory pressure system comprising an exhalation valve and an exhalation pressure sensor both in the exhaust line and further comprising an exhaust line flow sensor configured to measure a flow rate of the gas in the exhaust line.

10. An apparatus for delivering precise dosing of a Nitric Oxide containing gas for delivery by patient inhalation, the apparatus comprising:
a) a ventilation apparatus configured to deliver an oxygen containing gas to a patient interface for inhalation, the ventilation apparatus comprising,
A) a source of medical air,
B) a source of medical oxygen
C) an oxygen containing gas injection device in fluid communication with the source of medical air via a medical air supply line and the source of medical oxygen via a medical oxygen supply line,
D) one or more medical air pressure regulators in fluid communication with the medical air supply line and configured to control the pressure of the medical air in the medical air supply line,
E) one or more medical oxygen pressure regulators in fluid communication with the medical oxygen supply line and configured to control the pressure of the medical oxygen in the medical oxygen supply line,
F) a medical oxygen working pressure sensor configured to measure the pressure of a medical oxygen dose emitted from the oxygen containing gas injection device,
G) a medical air working pressure sensor configured to measure the pressure of a medical air dose emitted from the oxygen containing gas injection device,
H) an medical oxygen flow sensor configured to measure a flow rate of the medical oxygen dose emitted from the oxygen containing gas injection device,
I) an medical air flow sensor configured to measure a flow rate of the medical air dose emitted from the oxygen containing gas injection device,
J) an inspiratory gas tube in fluid communication with the oxygen containing gas injection device and configured to receive an injection of oxygen containing gas from the oxygen containing gas injection device,
K) a patient circuit pressure sensor configured to measure a gas pressure in the inspiratory gas tube,
b) a Nitric Oxide delivery apparatus configured to inject a Nitric Oxide containing gas into the oxygen containing gas prior to the patient interface, wherein the Nitric Oxide delivery apparatus comprises
A) a Nitric Oxide dose control system configured to inject a controlled amount of Nitric Oxide into the oxygen containing gas,
c) an exhaust line configured to receive an exhaled gas from a patient, a bypass flow of oxygen containing gas, or both, and to transport the exhaled gas to an exhaust vent,
d) a positive expiratory pressure system comprising an exhalation valve and an exhalation pressure sensor in the exhaust line,
e) an exhaust sampling line in fluid communication with the exhaust line prior to the exhaust vent and configured to receive a sample of a gas in the exhaust line comprising any exhaled gas,
f) a first electrochemical cell in fluid communication with the exhaust sampling line and configured to receive at least part of the exhaust gas sample and further configured to measure the amount of Nitric Oxide in the sampling portion of the exhaled gas,
g) a second electrochemical cell in fluid communication with the exhaust sampling line and configured to receive at least part of the exhaust gas sample and further configured to measure the amount of NO₂ in the sampling portion of the exhaled gas,
h) an exhaust line flow sensor configured to measure a flow rate of the gas in the exhaust line,
i) a patient interface in fluid communication with the inspiratory gas tube and the exhaust line,
j) a computer specifically programmed or a microprocessor specifically configured to execute the following functions:
A) calculate a Nitric Oxide dose provided to a patient by the Nitric Oxide delivery apparatus,
B) calculate an amount of NOx in the gas in the exhaust line comprising any exhaled gas.

11. The apparatus of claim 10, wherein the computer is further specifically programmed or a microprocessor is further specifically configured to calculate an amount of NO delivered to a patient based on the Nitric Oxide dose and the amount of NOx in the gas in the exhaust line.

12. The apparatus of claim 10, further comprising an exhaust gas sample mixing chamber between and in fluid communication with a) the exhaust sample line and b) the first and second electrochemical cells, wherein the exhaust gas sample mixing chamber is configured to form a substantially homogenous exhaust gas sample.

13. The apparatus of claim 10, further comprising an alarm system configured to be activated in response to a calculated amount of NOx in the exhaust line gas in excess of a pre-defined threshold amount.

14. The apparatus of claim 10, further comprising an exhaust gas mixing chamber between and in fluid communication with a) the exhaust line and b) the exhaust sampling line, wherein the exhaust gas mixing chamber is configured to form a substantially homogenous exhaust gas upstream from the exhaust sampling line.
